(19) United States
(12) Patent Application Publication
(10) Pub. No.: US 2004/0063906 A1
(43) Pub. Date: Apr. 1, 2004

(54) Pna analogues

(76) Inventors: Peter E. Nielsen, Kokkedal (DK); Ask Puschl, Frederiksberg (DK)

Correspondence Address: DARBY & DARBY P.C., P. O. BOX 5257, NEW YORK, NY, 10150-5257, US

(21) Appl. No.: 10/433,016

(22) PCT Filed: Nov. 23, 2001
(86) PCT No.: PCT/DK01/00779

(30) Foreign Application Priority Data
Nov. 24, 2000 (DK) PA 2000 01776
Mar. 6, 2001 (DK) PA 2001 00371
Jul. 16, 2001 (DK) PA 2001 01117

(51) Int. Cl. C07K 14/00

(52) U.S. Cl. 530/350; 530/324

(57) ABSTRACT

The present invention concerns peptide nucleic acid (PNA) sequences, which are modified in order to obtain novel PNA molecules with enhanced properties.

The present invention concerns new, stable peptide nucleic acid (PNA) oligomers.

### BACKGROUND OF THE INVENTION

Antisense agents offer a novel strategy in combating diseases, as well as opportunities to employ new chemical classes in the drug design.

Oligonucleotides can interact with native DNA and RNA in several ways. One of these is duplex formation between an oligonucleotide and a single stranded nucleic add. Another is triplex formation between an oligonucleotide and double stranded DNA to form a triplex structure.

Results from basic research have been encouraging, and antisense oligonucleotide drug formulations against viral and disease causing human genes are progressing through clinical trials. Efficient antisense inhibition of bacterial genes also could have wide applications; however, there have been few attempts to extend antisense technology to bacteria.

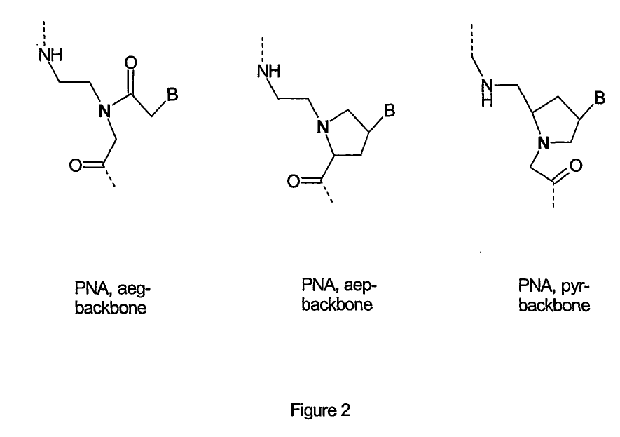

Peptide nucleic acids (PNA) are compounds that in certain respects are similar to oligonucleotides and their analogs and thus may mimic DNA and RNA. In PNA, the deoxyribose backbone of oligonucleotides has been replaced by a pseudo-peptide backbone (Nielsen et al. 1991 (1)) (FIG. 2). Each subunit, or monomer, has a naturally occurring or non-naturally occurring nucleobase attached to this backbone. One such backbone is constructed of repeating units of N-(2-aminoethyl)glycine linked through amide bonds. PNA hybridises with complementary nucleic acids through Watson and Crick base pairing and helix formation (Egholm et al. 1993 (2)). The Pseudo-peptide backbone provides superior hybridization properties (Egholm et al. 1993 (2)), resistance to enzymatic degradation (Demidov et al. 1994 (3)) and access to a variety of chemical modifications (Nielsen and Haaima 1997 (4)).

PNA binds both DNA and RNA to form PNA/DNA or PNA/RNA duplexes. The resulting PNA/DNA or PNA/RNA duplexes are bound with greater affinity than corresponding DNA/DNA or DNA/RNA duplexes as determined by Tm's. This high thermal stability might be attributed to the lack of charge repulsion due to the neutral backbone in PNA. In addition to increased affinity, PNA has also been shown to bind to DNA with increased specificity. When a PNA/DNA duplex mismatch is melted relative to the DNA/DNA duplex, there is seen an 8 to 20° C. drop in the Tm.

Furthermore, homopyrimidine PNA oligomers form extremely stable PNA₂-DNA triplexes with sequence complementary targets in DNA or RNA oligomers. Finally, PNA's may bind to double stranded DNA or RNA by helix invasion.

An advantage of PNA compared to oligonucleotides is that the PNA polyamide backbone (having appropriate nucleobases or other side chain groups attached thereto) is not recognised by either nucleases or proteases and are thus not cleaved. As a result, PNA's are resistant to degradation by enzymes unlike nucleic acids and peptides.

For antisense application, target bound PNA can cause steric hindrance of DNA and RNA polymerases, reverse transcription, telomerase and the ribosome's (Hanvey et al. 1992 (5), Knudsen eta. 1996 (6), Good and Nielsen 1998 (11, 12)), etc.

A general difficulty when using antisense agents is cell uptake. A variety of strategies to improve uptake can be envisioned and there are reports improved uptake into eukaryotic cells using lipids (Lewis et al. 1996 (7)), encapsulation (Meyer et al. 1998 (8)) and carrier strategies (Nyce and Metzger 1997 (9), Pooga et al, 1998 (10)).

WO 99/05302 discloses a PNA conjugate consisting of PNA and the transporter peptide transportan, which peptide may be used for transport cross a lipid membrane and for delivery of the PNA into interactive contact with intracellular polynucleotides.

U.S. Pat. No. 5,777,078 discloses a pore-forming compound which comprises a delivery agent recognising the target cell and being linked to a pore-forming agent, such as a bacterial exotoxin. The compound is administered together with a drug such as PNA.

As an antisense agent for microorganisms, PNA may have unique advantages. It has been demonstrated that PNA based antisense agents for bacterial application can control cell growth and growth phenotypes when targeted to Escherichia coli rRNA and mRNA (Good and Nielsen (11,12) and WO 99/13893).

However, none of these disclosures discuss ways of transporting the PNA across the bacterial cell wall and membrane.

Furthermore, for bacterial application, poor uptake is expected, because bacteria have stringent barriers against foreign molecules and antisense oligomer containing nucleobases appear to be too large for efficient uptake. The results obtained by Good and Nielsen (11,12)) indicate that PNA oligomers enter bacterial cells poorly by passive diffusion across the lipid bilayers.

U.S. Pat. No. 5,834,430 discloses the use of potentiating agents, such as short cationic peptides in the potentiation of antibiotics. The agent and the antibiotic are co-administered.

WO 96/11205 discloses PNA conjugates, wherein a conjugated moiety may be placed on terminal or non terminal parts of the backbone of PNA in order to functionalise the PNA. The conjugated moieties may be reporter enzymes or molecules, steroids, carbohydrate, terpenes, peptides, proteins, etc. It is suggested that the conjugates among other properties may possess improved transfer properties for crossing cellular membranes. However, WO 96/11205 does not disclose conjugates, which may cross bacterial membranes.

WO 98/52614 discloses a method of enhancing transport over biological membranes, e.g. a bacterial cell wall. According to this publication, biological active agents such as PNA may be conjugated to a transporter polymer in order to enhance the transmembrane transport. The transporter polymer consists of 6-25 subunits; at least 50% of which contain a guanidino or amidino sidechain moiety and wherein at least 6 contiguous subunits contain guanidino and/or amidino sidechains. A preferred transporter polymer is a polypeptide containing 9 arginine.

### SUMMARY OF THE INVENTION

The present invention relates to a novel peptide nucleic acid (PNA) oligomer and of PNA oligomers linked to a peptide characterized in that the single units of the oligomer consists of different amino acid backbones as shown in FIG. 2. The backbones are selected from aminoethylglycin (aeg), aminoethylprolyl (aep), aminoethylpyrrolidine (pyr) or from an amino acid different from aeg, aep or pyr (aa).

Accordingly, the present invention relates to a novel peptide nucleic acid (PNA) oligomer of from 4 to 25 monomers selected from the group consisting of aeg-PNA, pyr-PNA, aep-PNA and aa-PNA or of pyr-PNA units only.

PNA oligomers consisting of from 4 to 25 monomers of the present invention targeted to specific sequences of the messenger RNA of specific genes can be used as antisense reagents and drugs for down regulation of the expression of these genes in molecular biology and medicine. The PNA oligomers may be conjugated to carrier peptides to facilitate cellular uptake. Medical applications include treatment of bacterial and viral infections, cancer, metabolic diseases, immunological disorders etc.

PNA oligomers may also be used as hybridization probes in genetic diagnostics as exemplified by in situ hybridization, real time PCR monitoring and PCR modulation by “PNA-clamping”.

Finally, PNA oligomers that bind to targets in double stranded DNA by a variety of mechanisms (e.g. triplex binding, duplex invasion, triplex invasion and double duplex invasion) may be developed into antigene drugs by targeting specific sequences of specific genes. In this way the expression of the targeted gene can be inhibited (or in desired cases activated), and the level of a disease related gene product thereby regulated.

The present invention further concerns a new strategy for combating bacteria. It has previously been shown that antisense PNA can inhibit growth of bacteria. However, due to a slow diffusion of the PNA over the bacterial cell wall a practical application of the PNA as an antibiotic has not been possible previously. According to the present invention, a practical application in tolerable concentration may be achieved by modifying the PNA by linking a peptid or peptide-like sequence, which enhances the activity of the PNA.

The present invention further concerns a modified PNA molecule of formula (I):

Q—L—PNA (I)

wherein L is a linker or a bond;

Q is any amino add sequence and

PNA is a peptide nucleic acid oligomer with from 4 to 25 monomers selected from the group consisting of aeg-PNA, pyr-PNA, aep-PNA or aa-PNA, provided that the oligomer contains at least one pyr-PNA monomer group.

The peptide and the PNA oligomer are linked together as disclosed in the experimental part of PCT Publication WO 01/27261.

In one embodiment, The Peptide of the present invention contains from 2 to 60 amino acids. The amino acids can be negatively, non-charged or positively charged naturally occurring, rearranged or modified amino acids.

In a preferred embodiment of the invention the PNA oligomer contains from 6 to 12 oligomer units.

In a preferred embodiment of the invention the peptide contains from 2 to 18 amino acids, most preferred from 5 to 15 amino acids.

The peptide is linked to the PNA sequence via the amino (N-terminal) or carboxy (C-terminal) end.

In a preferred embodiment the peptide is linked to the PNA sequence via the carboxy end.

Within the present invention, the compounds of formula I may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, Berge, S. M. et al, 66, 1-19 (1977) (31) which are known to the skilled artisan.

Also intended as pharmaceutically acceptable acid addition salts are the hydrates, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

In another aspect of the invention the modified PNA molecules are used in the manufacture of medicaments for the treatment or prevention of infectious diseases or for disinfecting non-living objects.

In a further aspect, the invention concerns a composition for treating or preventing infectious diseases or disinfecting non-living objects.

In yet another aspect, the invention concerns the treatment or prevention of infectious diseases or treatment of non-living objects.

In yet a further aspect, the present invention concerns a method of identifying specific advantageous antisense PNA sequences, which may be used in the modified PNA molecule according to the invention.

### DETAILED DESCRIPTION OF THE INVENTION

Recently, the stabilisation of the PNA backbone by a prolyl unit has been introduced (D'Costa et al, 1999 (13)). The insertion of a cyclic ring leads to more stable structures, which are both rigid and cationic, and thus forming more stable triplexes with DNA. The PNA modified as described by D'Costa et al is designated aminoethylprolyl (aep) PNA (FIG. 2). A corresponding PNA wherein the cyclic ring is a pyrrolidine ring is designated pyrrolidine (pyr) PNA, also shown in FIG. 2. Finally, FIG. 2 shows the chemical structures of PNA with N-(2-aminoethyl)glycine (aeg). PNA, wherein the backbone is an amino acid different from the three structures as shown in FIG. 2, is designated aa-PNA.

Examples of preferred modified PNA molecules according to the invention are (Lys Phe Phe)₃Lys-L-PNA—wherein L designates an optional linker—and any subunits thereof comprising at least three amino acids. Preferred peptides are disclosed in PCT Publication WO 01/27261 including, but not limited to: (Lys Phe Phe)₃, (Lys Phe Phe)₂Lys Phe, (Lys Phe Phe)₂Lys, (Lys Phe Phe)₂, Lys Phe Phe Lys Phe, Lys Phe Phe Lys and Lys Phe Ph.

The PNA molecule is connected to the Peptide moiety through a direct binding or through a linker. A variety of linking groups can be used to connect the PNA with the Peptide.

Linking groups are described in WO 96/11205, WO 98/52614 and WO 01/27261, the content of which are hereby incorporated by reference.

Some linking groups may be advantageous in connection with specific combinations of PNA and Peptide.

The Peptide is normally linked to the PNA sequence via the amino or carboxy end. However, the PNA sequence may also be linked to an internal part of the peptide or the PNA sequence is linked to a peptide via both the amino and the carboxy end.

The modified PNA molecule according to the present invention comprises a PNA oligomer of a sequence, which is complementary to at least one target nucleotide sequence in a microorganism, such as a bacterium. The target may be a nucleotide sequence of any RNA, which is essential for the growth, and/or reproduction of the bacteria. Alternatively, the target may be a gene encoding a factor responsible for resistance to antibiotics. In a preferred embodiment, the functioning of the target nucleotide sequence is essential for the survival of the bacteria and the functioning of the target nucleic acid is blocked by the PNA sequence, in an antisense manner.

The binding of a PNA strand to a DNA or RNA strand can occur in one of two orientations, anti-parallel or parallel. As used in the present invention, the term complementary as applied to PNA does not in itself specify the orientation parallel or anti-parallel. It is significant that the most stable orientation of PNA/DNA and PNA/RNA is anti-parallel. In a preferred embodiment, PNA targeted to single strand RNA is complementary in an anti-parallel orientation.

In a another preferred embodiment of the invention a bis-PNA consisting of two PNA oligomers covalently linked to each other is targeted to a homopurine sequence (consisting of only adenine and/or guanine nucleotides) in RNA (or DNA), with which it can form a PNA₂-RNA (PNA₂-DNA) triple helix.

In another preferred embodiment of the invention, the PNA contains from 5 to 20 nucleobases, in particular from 7-15 nucleobases, and most particular from 8 to 12 nucleobases.

In another aspect, the present invention includes within its scope pharmaceutical compositions comprising, as an active ingredient, at least one of the compounds of the general formula I, or a pharmaceutically acceptable salt thereof together with a pharmaceutically acceptable carrier or diluent.

Pharmaceutical compositions containing a compound of the present invention may be prepared by conventional techniques, e.g. as described in Remington: The Science and Practice of Pharmacy, Gennaro, A. R (editor), 19^{th} Ed., 1995. The compositions may appear in conventional forms, for example capsules, tablets, aerosols, solutions, suspensions or topical applications.

Typical compositions include a compound of formula I or a pharmaceutically acceptable acid addition salt thereof, associated with a pharmaceutically acceptable excipient which may be a carrier or a diluent or be diluted by a carrier, or enclosed within a carrier which can be in the form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier, which may be in the form of an ampoule, capsule, sachet, paper, or other container. When the carrier serves as a diluent, it may be solid, semi-solid, or liquid material, which acts as a vehicle, excipient, or medium for the active compound. The active compound can be adsorbed on a granular solid container for example in a sachet Some examples of suitable carriers are water, salt solutions, alcohol's, polyethylene glycol's, polyhydroxyethoxylated castor oil, peanut oil, olive oil, gelatin, lactose, terra alba, sucrose, glucose, cyclodextrine, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents, thickeners or flavoring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or coloring substances and the like, which do not deleteriously react with the active compounds.

The route of administration may be any route, which effectively transports the active compound to the appropriate or desired site of action, such as oral, nasal, rectal, pulmonary, transdermal or parenteral e.g. depot, subcutaneous, intravenous, intraurethral, intramuscular, intranasal, ophthalmic solution or an ointment, the parenteral or the oral route being preferred.

If a solid carrier is used for oral administration, the preparation may be tabletted placed in a hard gelatin capsule in powder or pellet form or it can be in the form of a troche or lozenge. If a liquid carrier is used, the preparation may be in the form of a suspension or solution in water or a non-aqueous media, a syrup, emulsion or soft gelatin capsules. Thickeners, flavorings, diluents, emulsifiers, dispersing aids or binders may be added.

For nasal administration, the preparation may contain a compound of formula I dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrine, or preservatives such as parabenes. For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, cornstarch, and/or potato starch. A syrup or elixir can be used in cases where a sweetened vehicle can be employed.

In formulations for treatment or prevention of infectious diseases in mammals the amount of active modified PNA molecules used is determined in accordance with the specific active drug, organism to be treated and carrier of the organism. Such mammals include also animals, both domestic animals, e.g. household pets, and non-domestic animals such as wildlife. Usually, dosage forms suitable for oral, nasal, pulmonal or transdermal administration comprise from about 0.01 mg to about 500 mg, preferably from about 0.01 mg to about 100 mg of the compounds of formula I admixed with a pharmaceutically acceptable carrier or diluent.

In a still further aspect, the present invention relates to the use of one or more compounds of the general formula I, or pharmaceutically acceptable salts thereof for the preparation of a medicament for the treatment and/or prevention of infectious diseases.

In yet another aspect of the present invention, the present invention concerns a method of treating or preventing infectious diseases, which treatment comprises administering to a patient in need of treatment or for prophylactic purposes an effective amount of modified PNA according to the invention. Such a treatment may be in the form of administering a composition in accordance with the present invention. In particular, the treatment may be a combination of traditional antibiotic treatment and treatment with one or more modified PNA molecules targeting genes responsible for resistance to antibiotics.

In yet a further aspect of the present invention, the present invention concerns the use of the modified PNA molecules in disinfecting objects other than living beings, such as surgery tools, hospital inventory, dental tools, slaughterhouse inventory and tool, dairy inventory and tools, barbers and beauticians tools and the like.

References

1. Nielsen, P. E., Egholm, M., Berg, R. H. and Buchardt, O. Science (1991) 254, 1497-1500.

2. Egholm, M, Buchardt, O, Christensen, L, Behrens, C, Freier, S. M. Driver, D. A., Berg, R. H., Kim, S. K., Norden, B. and Nielsen, P. E. Nature (1993) 365, 566-568.

3. Demidov, V., Potaman, V. N., Frank-Kamenetskii, M. D., Egholm, M., Buchardt, O. Sönnichsen, H. S. and Nielsen, P. E. Biochem. Pharmacol. (1994) 48, 1310-1313.

4. Nielsen, P. E. and Haaima, G. Chemical Society Reviews (1997) 73-78.

5. Hanvey et al. Science (1992) 258,1481-5.

6. Knudsen, H. and Nielsen, P. E. Nucleic Acids Res. (1996) 24, 494-500.

7. Lewis, L. G. et al. Proc. Natl. Acad. Sci. USA (1996) 93, 3176-81.

8. Meyer, O. et al. J. Biol. Chem. (1998) 273, 15621-7.

9. Nyce, J. W. and Metzger, W. J. Nature (1997) 385 721-725.

10. Pooga, M. et al, Nature Biotechnology (1998) 16, 857-61.

11. Good, L. & Nielsen, P. E. Proc. Nati. Acad. Sci. USA (1998) 95, 2073-2076.

12. Good, L. & Nielsen, P. E. Nature Biotechnology (1998) 16, 355-358.

13. D'Costa, Moneesha, Vaijayant A. Kumar and Krishna N. Ganesh, Organic Letters, 1999, 1 (10), 1513-1516.

14. Nielsen, P. E.; Egholm, M.; Berg,.R. H.; Buchardt, O. Science 1991, 254, 1497-1500.

15. Uhlmann, E.; Peyman, A.; Breipohl, G.; Will, D. W. Angew. Chem. Int Ed. 1998, 37, 2796-2823.

16. Larsen, H. J.; Bentin, T.; Nielsen, P. E. Biochim. Biophys. Acta 1999, 1489, 159-166.

17. (a) Püschl, A.; Boesen, T.; Zuccarello, G.; Dahl, O.; Nielsen, P. E. “Peptide Nucleic Acids with a constrained Cyclic backbone”. Poster 33, shown at the Sixth International Symposium: Solid phase synthesis & combinatorial libraries, York England, 31 Aug. 1999. (b) Püschl, A.; Boesen, T.; Zuccarello, G.; Dahl, O.; Nielsen, P. E. Manuscript submitted July 2000.

18. (a) Eriksson, M.; Nielsen, P. E. Nature Structural Biology, 1996, 3, 410. (b) Brown, S. C.; Thomson, S. A.; Veal, J. M.; Davies, D. G. Science 1994, 265, 777.

19. Hyrup, B.; Egholm, M.; Buchardt, O.; Nielsen, P. E. Bioorganic & Med. Chem. Lett. 1996, 6, 1083-1088.

20. Lowe, G.; Vilaivan, T. J. Chem. Soc., Perkin Trans. 1, 1997, 539-546.

21. Peterson, M. L.; Vince, R. J. Med. Chem. 1991, 34, 2787-2797.

22. Nakamura, T.; Matsuyama, H.; Kamigata, N.; lyoda, M. J. Org. Chem. 1992, 57, 3783-3789.

23. Altmann, K.-H.; Hüsken, D.; Cuenoud, B.; Garcia-Echeverria, C. Bioorganic & Med. Chem. Lett. 2000, 10, 929-933.

24. Saito, S.; Nakajima, H.; Inaba, M.; Moriwake, T. Tetrahedron Left. 1989, 30, 837-840.

25. Thomson, S. A.; Josey, J. A.; Cadilla, R.; Gaul, M. D.; Hassman, C. F.; Luzzio, M. J.; Pipe, A. J.; Reed, K. L.; Ricca, D. J.; Wiethe, R. W.; Noble, S. A. Tetrahedron, 1995, 51, 6179-6194.

26. Watkins, B. E.; Rapoport, H. J. Org. Chem. 1982, 47, 4471.

27. Chenon, M. T.; Pugmire, R. J.; Grant, D. M.; Panciza, R. P.; Townsend, L. B. J. Am. Chem. Soc. 1975,4627-4636.

28. Koch, T.; Hansen, H. F.; Andersen, P.; Larsen, T.; Batz, H. G.; Otteson, K.; Ørum, H. J. Peptide Res. 1997, 49, 80-88.

29. Hickman, D. T.; King, P. M.; Cooper, M. A.; Slater, J. M.; Micklefield, J. Chemm. Commun. 2000, 2251-2252.

30. Kumar. V.; Pallan, P. S.; Meena, Ganesh, K. N. Org. Leff. 2001, 3,1269-1272. (d) Bai, J.-Q.; Li, Y.; Liu, K.-L. Chinese J. Chem., 2001, 19, 276-281.

31. Pharmaceutical Science, Berge, S. M. et al, 66, 1-19 (1977).

32. Ray, Arghya and Bengt Norén. The FASEB Journal, 2000, 14, p. 1041-1060.

### EXAMPLES

### Example 1

Preparation of Adenine(A) Monomers

A new conformationally restricted PNA adenine monomer has been synthesized in 13 steps from cis-4-hydroxy-D-proline. A fully modified adenine decamer displayed improved binding affinity towards complementary DNA and RNA strands as compared to the parent PNA adenine decamer.

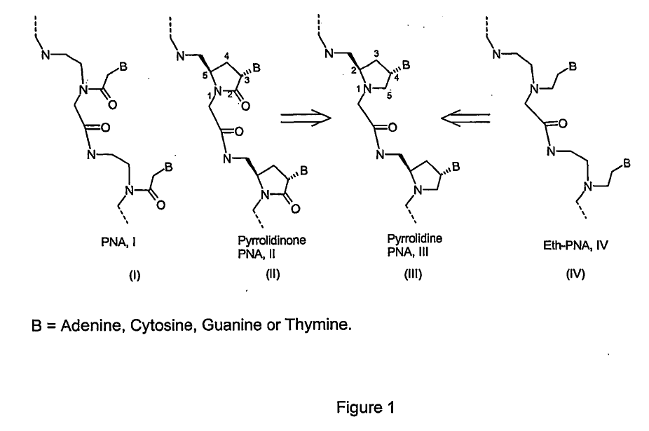

Peptide nucleic acid (PNA) is a DNA mimic in which the nucleobases are linked to a N-(2-aminoethyl)glycine backbone through methylene carbonyl linkers (FIG. 1) (14). PNA binds to DNA and RNA with high affinity and specificity (15). The antisense property of PNA has recently been reviewed (16). In this context it is the binding affinity of PNA towards RNA that is important. PNA/DNA and PNA/RNA duplex formation is accompanied by a decrease in entropy. This entropy loss could be reduced, by using a more rigid PNA analogue.

We have recently designed and synthesized such an analogue: The Pyrrolidinone PNA (FIG. 1, B=Adenine) (17). In this analogue, the carbonyl group of the linker is forced to point towards the COOH terminus of the backbone. This approximates the conformation of the PNA strands found in the 3-dimensional structures of PNA/DNA and PNA/RNA duplexes (18). As opposed to PNA, which is achiral, two new stereocenters are introduced in each monomer units in the pyrrolidinone PNA. By synthesizing all four possible stereoisomeric monomer building blocks and incorporating them into PNA oligomers it was found that the (3S, 5R) isomer was the best isomer. A fully modified (3S, 5R)-pyrrolidinone adenine decamer displayed a Tₘ depression per modification of only 1° C. as compared to unmodified PNA against r(U)₁₀.

However, a larger destabilisation (ΔTₘ/mod −3.5° C.) against complementary RNA was seen when the (3S, 5R)-pyrrolidinone analogue was incorporated once into a decamer PNA oligomer.

Inspired by the recent publication by D'Costa et. al. (13) of the aminoethylprolyl PNA (aep-PNA, FIG. 2), we decided to synthesize the reduced analogue of the pyrrolidinone PNA (II): The pyrrolidine PNA (III) (FIG. 1, B=Adenine). III (and aminoethylprolyl PNA) is also an analogue of the flexible Eth-PNA IV (19). Incorporation of IV (FIG. 1, B=Thymine) into PNA oligomers were shown to destabilize duplex and triplex formation considerably. Unfortunately, no fully modified oligomer of IV was syntehesized.

Monomer synthesis. The synthesis of the protected (2R, 4S) adenine pyrrolidinine monomer A12 is shown in the scheme. cis-4-Hydroxy-D-proline was synthesized by the epimerisation of trans-4-hydroxy-L-proline in 22% yield as described (20). The secondary amine was Boc protected in 81% yield as described (21). N-Boc-cis-4-hydroxy-D-proline methyl ester A1 is usually prepared by the diazomethane procedure (22). Instead we prepared A1 in 99% yield by alkylating the cesium salt of the acid with Mel in DMF. A3 was prepared from A1 via A2 as described (23). The azide A5 was prepared via the mesyl compound A4. The Boc protecting group was cleaved with TFA, and the resulting secondary amine A6 was alkylated with methyl bromoacetate in the presence of DIEA. Reductive Boc amination (24), followed by standard TBAF cleavage of the TBDPS (tert-butyldiphenylsilyl) group produced the novel pyrrolidine backbone A9. At this point it was planned to introduce the adenine base under Mitsunobu conditions. However, all attempts to substitute the secondary hydroxy group by adenine using DEAD and PPh₃ failed.

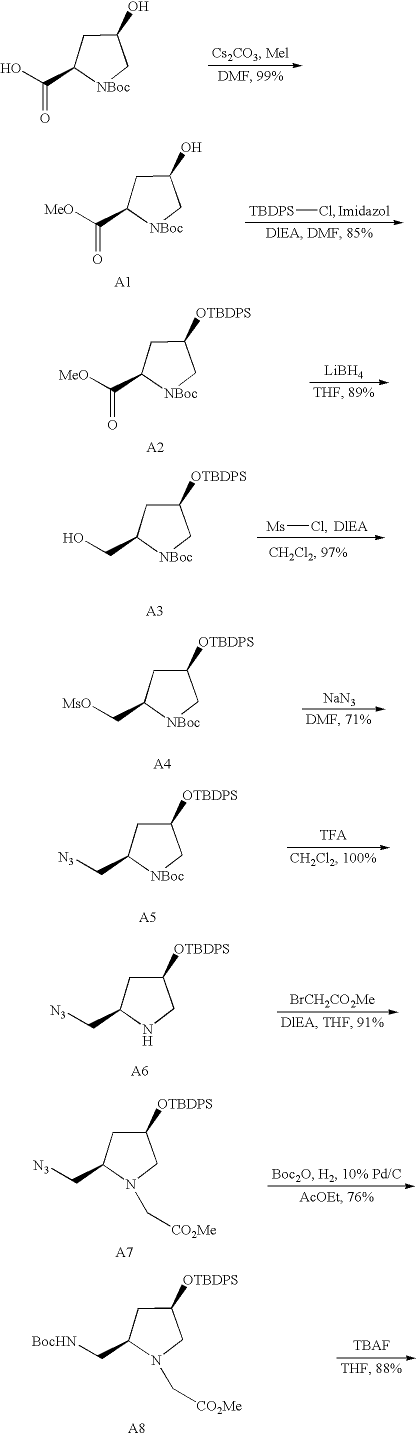

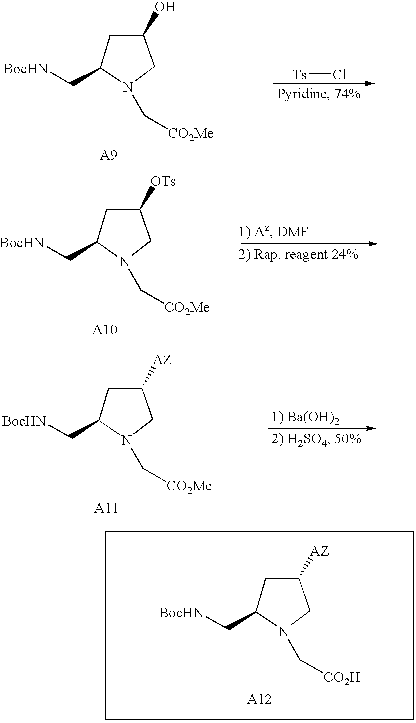

This is probably due to the presence of the tertiary amine since adenine is easily attached to the corresponding pyrrolidinone derivative using Mitsunobu conditions (17). Instead adenine was introduced, by converting A9 into the tosyl compound A10 and then displacing the tosyl group with benzyloxycarbonyl protected adenine. Using adenine instead of benzyloxycarbonyl protected adenine (25) gave very low yield. For yet unknown reasons the Z group was lost during the reaction but adenine was readily re protected using Rapoports reagent thus producing A11 (26). ¹³C-NMR proved that the correct N9 isomer was obtained (27). Finally, A12 was synthesized by cleaving the methyl ester with Ba(OH)₂ and then precipitating BaSO₄ with H₂SO₄. In this way A12.H₂SO₄ was recovered by lyophilizing the aq phase.

In detail the monomers were synthesized in the following way:

General Information. ¹H and ¹³C NMR spectra were taken in CDCl₃ at 300 MHz and 75.0 MHz respectively unless specified otherwise. Chemical shifts are reported in parts per million using the solvent resonance internal standard (chloroform, 7.24 and 76.9 ppm). Pyridine, CH₂Cl₂, DMF and CH3CN were dried over 4 Å molecular sieves. THF was distilled from sodium. Reaction where carried out under nitrogen unless otherwise noted. Manual Boc-PNA Solid phase synthesis was carried out in a glass reactor. The references refer to those given in the Letter.

Preparation of compound A1. Cs₂CO₃ (3.42 g, 10.5 mmol) was added to a stirred solution of N-Boc-cis-4-hydroxy-D-proline (2.31 g, 10.0 mmol) in dry DMF (36 ml). The reaction mixture was stirred 15 min after which Mel (0.75 ml, 12.0 mmol) was added dropwise. The reaction mixture was stirred overnight and then filtered through celite. The DMF was evaporated off and the residue was partitioned between sat NaHCO₃ (100 ml) and AcOEt (200 ml). The organic phase was washed with brine (2×100 ml), dried (MgSO₄) and evaporated in vacuo. Yield: 2.42 g (99%) off A1 as a white solid. [α]_{D}²⁰=65.0 (c 1, EtOH) (Litt:⁹ [α]_{D}²⁵=63.8(c 2.21, EtOH)).

Preparation of compound A2 (23). Imidazol (1.44 g, 21.1 mmol), DIEA (2.5 ml, 14.4 mmol) and then tert-butyl-diphenylsilyl chloride (3.75 ml, 14.4 mmol) were added to a stirred solution of A1 (2.35 g, 9.60 mmol) in dry DMF (19 ml). The reaction mixture was stirred overnight and then filtered through celite. The DMF was evaporated off and the residue was partitioned between half sat NaHCO₃ (100 ml) and AcOEt (100 ml). The organic phase was washed with brine (50 ml), 10% citric acid (50 ml), brine (2×50 ml), and then dried (MgSO₄) and evaporated in vacuo. The crude material (6 g) was purified by chromatography (AcOEt:Hexan 1:9). Yield: 3.98 9 (85%) of A2 as a white solid. NMR complicated by cis-trans isomeri around the Boc group: ¹H NMR (CDCl₃) δ 7.65-7.62 (m, 4H), 7.42-7.38 (m, 6H), 4.31-4.24 (m, 2×H), 3.75 (s, 3H), 3.60-3.38 (m, 2H), 2.23-2.16 (m, 2H), 1.45 and 1.42 (2×s, 9H), 1.07 and 1.04 (2×s, 9H). ¹³C NMR (CDCl₃) δ 174.9, 172.7, 172.3, 154.2, 153.5, 135.6, 135.5, 134.6, 133.4, 133.2, 133.0, 129.7, 129.4, 127.6, 127.5, 79.8, 71.5, 70.4, 57.6, 57.2, 54.2, 53.8, 52.0, 51.9, 39.1, 38.3, 28.3, 28.2, 26.6, 26.4, 18.8. FAB⁺MS: 484.33 (MH⁺). Calcd for C₂₇H₃₇NO₅Si: C, 67.05; H, 7.71; N, 2.90. Found: C, 66.90; H, 7.74; N, 2.94.

Preparation of compound A3 (23). LiBH₄ (23.5 ml, 2.0 M in THF) was slowly added to a stirred solution of A2 (18.2 g, 37.6 mmol) in dry THF (100 ml) at 0° C. The reaction mixture was allowed to warm to rt and then stirred 8 h. The reaction was quenched at 0° C. by the addition of H₂O (150 ml), followed by the slow addition of 1 M HCl (75 ml). The acidic solution was extracted with AcOEt (3×200 ml). The combined organic phases were washed with brine (100 ml), sat NaHCO₃ (100 ml), brine (100 ml) and dried (MgSO₄) and evaporated. The crude material (17.6 g) was purified by chromatography (1-10% MeOH in CH₂Cl₂). Yield: 15.17 g (89%) off A3 as, a white foam. NMR complicated by cis-trans isomeri around the Boc group: ¹H NMR (CDCl₃) δ 7.65-7.62 (m, 4H), 7.45-7.37 (m, 6H), 4.28 (m, 1H), 3.97 (m, 1H), 3.86 (m, 1H), 3.75 (m, 1H), 3.40-3.25 (m, 2H), 2.70 (br. s, 1H), 2.08 (m, 1H), 1.80-1.60 (m, 1H), 1.44 (s, 9H), 1.07 (s, 9H). FAB⁺MS: 456.37 (MH⁺).

Preparation of compound A4. DIEA (8.7 ml, 50.1 mmol) and then methanesulfonyl chloride (3.1 ml, 40.0 mmol), was added to a stirred solution of A3 (15.2 g, 33.4 mmol) in dry CH₂Cl₂ (170 ml) at 0° C. The reaction mixture stirred at 0° C. 40 min and then quenched by the addition of half sat NaHCO₃ (200 ml). The layers were separated and the aq layer was extracted with CH₂Cl₂ (2×150 ml). The combined organic phases were washed with brine (100 ml), 10% citric acid (2×100 ml), brine (100 ml), and then dried (MgSO₄) and evaporated. Yield: 17.2 g (97%) off A4 as, a yellow foam. NMR complicated by cis-trans isomeri around the Boc group: ¹H NMR (CDCl3) 3 7.67-7.61 (m, 4H), 7.46-7.40 (m, 6H), 4.56 (m, 1H), 4.50-4.38 (m, 2H), 4.06 (m, 1H), 3.50-3.00 (m, 2H), 3.01 (s, 3H), 2.10-1.98 (m, 2H), 1.48 and 1.45 (2×s, 9H), 1.07 (s, 9H). FAB⁺MS: 534.20 (MH⁺).

Preparation of compound A5. NaN₃ (10.5 g, 162 mmol) was added to a stirred solution of A4 (17.2 g, 32.3 mmol) in dry DMF (160 ml) at room temperature. The reaction mixture was stirred at 90° C. 4 h after which the DMF was evaporated off. The residue was partitioned between half sat NaHCO₃ (100 ml) and AcOEt (200 ml). The aq phase was extracted with more AcOEt (200 ml). The combined organic phases were washed with brine (2×100 ml), dried (Na₂SO₄) and evaporated. The crude product (15.2 g) was purified by chromatography (AcOEt:Hexane 1:4). Yield: 10.97 g (71%) off A5 as a white solid. NMR complicated by cis-trans isomeri around the Boc group: ¹H NMR (CDCl₃) δ 7.59-7.54 (m, 4H), 7.38-7.30 (m, 6H), 4.24 (br. s, 1H), 3.80 (br. s, 1H), 3.57 (br. s, 1H), 3.40-3.10 (m, 2H), 2.00-1.90 (m, 2H), 1.38 (s, 9H), 0.99 (s, 9H). ¹³C NMR (CDCl₃) δ 153.9, 135.6, 133.0, 129.8, 127.7, 80.0, 71.2, 55.9, 54.8, 53.8, 52.6, 37.3, 36.5, 28.3, 26.7, 18.8. FAB⁺MS: 481.32 (MH⁺).

Preparation of compound A6. TFA (4.6 ml, 58 mmol) was added to a stirred solution of A5 (2.14 g, 4.45 mmol) in dry CH₂Cl₂ (4.6 ml) at 0° C. The ice bath was removed and the reaction mixture was stirred at room temperature 30 min. The reaction was quenched by the slow addition of sat NaHCO₃ (65 ml). The layers were separated and the aq phase was extracted with CH₂Cl₂ (2×100 ml). The combined organic phases were dried (MgSO₄) and evaporated. Yield: 1.70 g (100%) of A6 as an oil. ¹H NMR (CDCl₃) δ 7.80-7.63 (m, 4H), 7.45-7.39 (m, 6H), 4.40 (m, 1H), 3.60 (br. s, 1H), 3.49-3.44 (m, 2H), 3.30 (m, 1H), 3.00-2.80 (m, 2H), 2.01 (m,₁H), 1.60 (m, 1H), 1.07 (s, 9H). C¹³C NMR (CDCl₃) δ 135.53, 135.50, 134.7, 135.5, 129.7, 127.6, 127.4, 73.5, 57.1, 55.1, 54.5, 38.6, 26.7, 18.9. FAB⁺MS: 381.49 (MH⁺).

Preparation of compound A7. DIEA (4.69 ml, 27.0 mmol) and then methyl bromoacetate (2.35 ml, 24.8 mmol) was added to a stirred solution of A6 (8.78 g, 22.5 mmol) in dry THF (45 ml) at ° C. The ice bath was removed and the reaction mixture was stirred at room temperature 4 h and then filtered through celite. The solvent was evaporated off and the crude product was purified by chromatography (AcOEt:Hexane 1:4). Yield: 9.31 g (91%) of A7 as a clear oil. ¹H NMR (CDCl₃) δ 7.75-7.67 (m, 4H), 7.47-7.36 (m, 6H), 4.43 (m, 1H), 3.67 (s, 3H), 3.57 (s, 2H), 3.54-3.36 (m, 2H), 3.14-3.11 (m, 2H), 2.84-2.79 (m,1H), 2.19-2.05 (m, 1H), 1.80-1.76 (m, 1H), 1.09 (s, 9H). FAB⁺MS: 453.22 (MH⁺).

Preparation of compound A8. A degassed solution of A7 (1.50 g, 3.31 mmol), Boc₂O (1.45 g, 6.62 mmol) and 10% Pd/C (0.23 g) in AcOEt (33 ml) was hydrogenated at room temperature overnight using balloon technique. Occasionally the nitrogen that developed was lead out through a needle outlet. The catalyst was removed by filtering the solution through celite. The solvent was evaporated off and the crude product was purified by chromatography (1-10% MeOH in CH₂Cl₂). Yield: 1.32 g (76%) of A8 as a clear oil. ¹H NMR (CDCl₃) δ 7.69-7.63 (m, 4H), 7.45-7.34 (m, 6H), 5.43 (br. s, 1H), 4.30 (br. s, 1H), 3.66 (s, 3H), 3.60-3.40 (m, 1H), 3.40-3.00 (m, 5H), 2.68 (m, 1H), 2.15 (m, 1H), 1.82 (m, 1H), 1.43 (s, 9H), 1.08 (s, 9H). ¹³C NMR (CDCl₃) δ 171.2, 156.5, 135.3, 133.5, 129.6, 127.5, 78.9, 71.7, 61.6, 60.1, 52.8, 51.4, 41.6, 38.1, 28.3, 26.9, 18.9. FAB⁺MS: 527.32 (MH⁺). Calcd for C₂₉H₄₂N₂O₅Si: C, 65.56; H, 8.08; N, 5.27. Found: C, 65.64, 8.62, 5.41.

Preparation of compound A9. A 1 M solution of TBAF in THF (16.3 ml, 16.3 mmol) was added to a stirred solution of A8 (7.18 g, 13.6 mmol) in THF (70 ml) at room temperature. The reaction mixture was stirred 4 h at room temperature and then quenched by the addition of ¼ sat NH₄Cl (200 ml) and CH₂Cl₂ (250 ml). Th layers were separated and the aq phase was extracted with more CH₂Cl₂ (250 ml) and AcOEt (2×250 ml). The combined organic phases were dried (Na₂SO₄) and the solvent was evaporated off. The crude product (12.0 g) was purified by chromatography (1-10% MeOH in CH₂Cl₂). Yield: 3.47 g (88%) of A9 as a clear oil. ¹H NMR (CDCl₃) δ 5.51 (br. s, 1H), 4.23 (br. s, 1H), 3.64 (s, 3H), 3.60-3.20 (m,4H), 3.10-3.00(m, 2H), 2.90-2.85 (m, 1H), 2.71-2.66 (m, 1H), 2.27-2.17 (m, 1H), 1.67-1.61 (m, 1H), 1.37 (s, 9H). ¹³C NMR (CDCl₃) δ 171.4, 156.5, 78.9, 69.7, 62.4, 53.0, 51.5, 41.3, 37.8, 28.2. HR FAB⁺MS: 289.1771 (MH⁺) (Calcd for C₁₃H₂₅N₂O₅: 289.1763).

Preparation of compound A10. p-toluenesulfonyl chloride (1.64 g, 8.62 mmol) was added to a stirred solution of A9 (1.24 g, 4.31 mmol) in dry pyridine (10.8 ml). The orange reaction mixture was stirred overnight at room temperature and then quenched by the addition of CH₂Cl₂ (100 ml) and sat NaHCO₃ (50 ml). The organic phase was extracted with more sat NaHCO₃ (50 ml), washed with brine (50 ml), and dried (Na₂SO₄). The solvent was evaporated off and the crude product was purified by chromatography (AcOEt:Hexane 1:1). Yield: 1.42 g (74%) of A10 as a clear oil. ¹H NMR (CDCl₃) δ 7.75 (d, J=8.5 Hz, 2H), 7.30 (d, J≦8.8 Hz, 2H), 5.14 (br. s, 1H), 4.96 (br. s, 1H), 3.65 (s, 3H), 3.42 (m, 2H), 3.26 (m, 2H), 3.06-2.93 (m, 3H), 2.41 (s, 3H), 2.33-2.24 (m, 1H), 1.84-1.79 (m, 1H), 1.41 (s, 9H). ¹³C NMR (CDCl3) δ 170.4, 156.2, 144.6, 133.7, 129.7, 127.5, 79.7, 79.1, 60.3, 58.7, 51.9, 51.5, 41.0, 35.0, 28.1, 21.4. FAB⁺MS: 443.21 (MH⁺).

Preparation of compound A11. 6-N-(Benzyloxycarbonyl)adenine (404 mg, 1.5 mmol), K₂CO₃ (186 mg, 1.35 mmol) and Cs₂CO₃ (49 mg, 0.15 mmol) was stirred in dry DMF (2 ml) 5 min at room temperature. A solution of A10 (662 mg, 1.50 mmol) in dry DMF (4 ml) was added dropwise and the suspension was stirred at room temperature 1 h. The brown solution was further stirred at 80° C. 1.5 h and then 1.5 h at room temperature. The DMF was evaporated off and the crude product was purified by chromatography (6-15% MeOH in CH₂Cl₂ containing 0.5% DIEA). Yield: 278 mg of the Boc protected monomer adenine methylester. ¹³C NMR and FAB⁺MS showed that the benzyloxycarbonyl group had been lost: ¹³C NMR (CDCl₃) δ 170.9, 156.0, 155.6, 152.3, 149.3, 138.6, 119.3, 78.9, 60.4, 57.9, 52.1 and 51.9, 51.3, 49.5, 41.3, 34.1, 28.0. FAB⁺MS: 406.34 (MH⁺). This intermediate (278 mg, 0.69 mmol) was dissolved in dry CH₂Cl₂ (5 ml). N-Benzyloxycarbonyl-N′-methylimidazolium triflate (757 mg, 2.1 mmol) was added and the solution was stirred at room temperature overnight. The reaction was diluted by adding more CH₂Cl₂ (50 ml) and then quenched by adding half sat NaHCO₃ (25 ml). The layers were separated and the aq phase was extracted with CH₂Cl₂ (50 ml) and AcOEt (50 ml). The combined organic phases were dried (Na₂SO₄) and th solvent was evaporated off. The crude product (781 mg) was purified by chromatography (AcOEt:M OH 9:1). Yield: 195 mg (24%) of A11 as a white foam. ¹H NMR (CDCl₃) δ 10.0-9.8 (br. s, 1H), 8.69 (s, 1H), 8.01 (s, 1H), 7.40-7.26 (m, 5H) 5.24 (s, 2H), 5.14 (m, 1H), 4.97 (m, 1H), 3.67 (s, 3H), 3.62-3.35 (m, 5H), 3.06 (m, 2H), 2.28 (m, 2H), 1.41 (s, 9H). ¹³C NMR (CDCl₃) δ 170.9, 156.1, 152.2, 151.2, 149.5, 141.6, 135.2, 128.3, 128.24, 128.21, 122.1, 79.2, 67.4, 60.5, 57.8, 52.5, 52.1, 51.5, 41.1, 33.9, 28.1. HR FAB^{+MS:} 540.2586 (MH⁺) (Calcd for C₂₆H₃₄N₇O₆: 540.2570). Calcd for C₂₈H₃₃N₇O₆.0.25 H₂O: C, 57.39; H, 6.22; N,18.02. Found: C,57.71; H, 6.08; N, 17.37.

Preparation of compound A12. A solution of Ba(OH)₂.8H₂O (166 mg) in H₂O (5 ml) was added dropwise to A11 (190 mg, 0.35 mmol) dissolved in THF (5 ml) at 0° C. The ice bath was removed and the reaction mixture was stirred at room temperature 20 min. More H₂O (6 ml) was added and the THF was evaporated off. pH was adjusted to 2.3 by adding 4 N H₂SO₄ (0.35 ml) to the unclear solution. BaSO₄ was removed by centrifugation. The acidic solution was decanted and then lyopholized. The lyophilization was repeated from MeOH (1.2 ml) and H₂O (12 ml) to produce 86 mg (50%) of A12.H₂SO₄ as a powder. ¹H NMR (CDCl₃) peaks shows considerable broadening probably due to the presence of H2SO₄: δ 8.6 (2×br. s, 2×1H), 7.4-7.0 (m, 5H), 5.6-5.4 (br. s, 1H), 5.3-5.0 (m, 3H), 4.6-3.4 (m, 7H), 2.6-2.4 (m, 2H), 1.27 (s, 9H). Pure on Tlc (Butanol:Acetic acid: H2O 4:1:1) R_{f}=0.41 (UV, ninhydrin reactive). 92% pure on RP-HPLC. HR FAB^{+MS:} 526.2405 (MH⁺) (Calcd for C₂₅H₃₂N₇O₆: 526.2414).

### Example 2

Preparation of Adenine (A) Oligomers

In order to evaluate the binding affinity of the pyrrolidine PNA analogue three PNA dodecamers were synthesized (28):

| | |
|---|---|
| PNA 2005: | H-TAC-TCA-TAC-TCT-LysNH₂ |
| PNA 2075: | H-TAC-TCA*-TAC-TCT-LysNH₂ |
| PNA 2104: | H-TAC-TCA#-TAC-TCT-LysNH₂ |

A*=(3S, 5R) pyrrolidone PNA monomer

A#=(2R, 4S) pyrrolidine PNA monomer (A12)

Solid phase synthesis of H-TAC-TCA#-TAC-TCT-LysNH₂ (PNA 2104). This dodecamer was synthesized by the usual in situ neutralization method using HBTU and DIEA on a Boc-Lys-(2-Cl-Z)-MBHA-PS resin (25 mg, loading 0.12 mmol/g) (28). The novel monomer A# (6 mg, 11 μmol) was dissolved in DMF (140 μL). DIEA (8 μL, 45 μmol) was added and this solution was added to HBTU (4 mg, 10 μmol). The solution was preactivated 2 min and then added to the resin (3 μmol). The coupling reaction was allowed to proceed for 2.5 h before the activated solution was drained out. A small amount of beads were subjected to the Kaiser test, which produced a yellow color indicating complete reaction. Synthesis and cleavage (TFA:TFMSA:thioanisole:m-cresol 3:1:0.5:0.5) was continued the usual way (28). After ether precipitation, the crude PNA was purified by RP-HPLC. Yield: 1.2 mg (12%). MALDI-MS: 3306 (Calcd for MH⁺: 3303). Pure on RP-HPLC.

Solid phase synthesis of H-(A#)₁₀-LysNH₂ (PNA 2110). This decamer was synthesized as described for PNA 2104. Yield: 4.4 mg (51%). MALDI-MS: 2873 (Calcd for MH⁺: 2873). Pure on RP-HPLC.

Binding affinity. The binding affinity towards complementary RNA and DNA oligomers was measured by obtaining the Tₘ-curves (Table 1). As expected, incorporation of both the pyrrolidinone and the pyrrolidine analogue into the PNA strand results in destabilization against DNA and RNA compared to unmodified PNA (entry 1 vs. 2 and 3). Surprisingly a larger destabilization in the affinity towards DNA and RNA in the case of the pyrrolidine analogue (entry 3) as compared to the pyrrolidinone analogue (entry 2) was detected.

TABLE 1
Melting Temperatures (Tₘ values)^{a}

| Entry | Sequence | DNA | RNA |
|---|---|---|---|
| 1 | PNA 2005 | 49.5 | 59.5 |
| 2 | PNA 2075 | 41.0 | 53.8 |
| 3 | PNA 2104 | 28.5 | 44.5 |

^{a}Tₘ = melting temperature (measured in medium salt buffer: 100 mM NaCl, 10 mM phosphate, 0.1 mM EDTA, pH = 7.0). Heating rate: 1 K/min. UV absorbance measured at 254 nm.

A fully modified decamer (PNA 2110) was synthesis d:

| | |
|---|---|
| PNA 186: | H-Gly-(A)₁₀-NH₂ |
| PNA 2020: | H-(A*)₁₀-LysNH₂ |
| PNA 2110: | H-(A#)₁₀-LysNH₂ |

TABLE 2
Melting Temperatures (Tₘ Values)^{a}

| Entry | Sequence | 5′-d(T)₁₀ | 5′-r(U)₁₀ |
|---|---|---|---|
| 1 | PNA 186 | 55.5 (55.5) | 35.5 (35.5) |
| 2 | PNA 2020 | —^{b} | 25.5 |
| 3 | PNA 2110 | 79.0 (41.0) | 60.0 (45.5) |

^{a}Tₘ = melting temperature (measured in medium salt buffer: 100 mM NaCl, 10 mM phosphate, 0.1 mM EDTA, pH = 7.0). Heating rate: 1 K/min. UV absorbance measured at 254 nm.
^{b}Not a sigmoid melting curve. Values in parentheses are Tₘ for the cooling curves.

As can be seen (Table 2), the fully modified pyrrolidine decamer have high affinity towards DNA (ΔTₘ/mod=+2.5° C.) and RNA (ΔTₘ/mod=+2.5° C.) (compare entry 1 and 3). As opposed to the parent PNA 186, significant hysteresis was detected both against DNA and RNA, in the case of PNA 2110. Obtaining the melting curves at pH 9 instead of pH 7 in the case of PNA 2110 only lowered the Tₘ values about 3.5° C. against DNA and 1.5° C. against RNA (not shown).

The complex between PNA 2110 and DNA was further evaluated. UV-titration showed the complex between PNA 2110 and 5′-d(T)₁₀ to be a 1:2 complex. Furthermore, the recognition between PNA 2110 and 5′-d(T)₁₀ was shown to be sequence specific (Table 3).

TABLE 3
Melting Temperatures (Tₘ Values)^{a}

| entry | sequence | X = T^{b} | X = A^{b} | X =C^{b} | X = G^{b} |
|---|---|---|---|---|---|
| 1 | PNA 186 | 55.5 | nd | 36.5 (36.5) | 40.5 (40.5) |
| | | (55.5) | | | |
| 2 | PNA 2110 | 79.0 | 67.5 (35.5) | 71.0 (36.0) | 68.5 (37.0) |
| | | (41.0) | | | |

^{a}Tₘ = melting temperature (measured in medium salt buffer: 100 mM NaCl, 10 mM phosphate, 0.1 mM EDTA, pH) 7.0). Measured from 90 to 15° C.
Heating rate: 1 K/min.
UV absorbance measured at 254 nm.
nd = not determined.
Values in parentheses are Tm for the cooling curves.
^{b}DNA target 5′-d(TTT-TXT-TTT-T)-3′.

not determined. Values in parentheses are Tm for the cooling curves. ^{b} DNA target: 5′-d(TTT-TXT-TTT-T)-3′.

In conclusion we have designed and synthesized a novel highly soluble PNA analogue: The pyrrolidine PNA analogue. The preliminary Tₘ data indicates that this analogue has strong affinity towards DNA and RNA.

### Example 3

Preparation of Thymine (T) Monomers

General Information. ¹H and ¹³C NMR spectra were taken in DMSO-d₆ at 400 MHz and 100.6 MHz respectively unless specified otherwise. Chemical shifts are reported in parts per million using the solvent resonance internal standard (dimethylsulfoxid: 2.50 and 39.6 ppm. Chloroform: 7.24 and 76.9 ppm). Pyridine, CH₂Cl₂, DMF and CH₃CN were dried over 4 Å molecular sieves. THF was distilled from sodium. Reactions were carried out under nitrogen unless otherwise noted. Manual Boc-PNA Solid phase synthesis was carried out in a glass reactor.

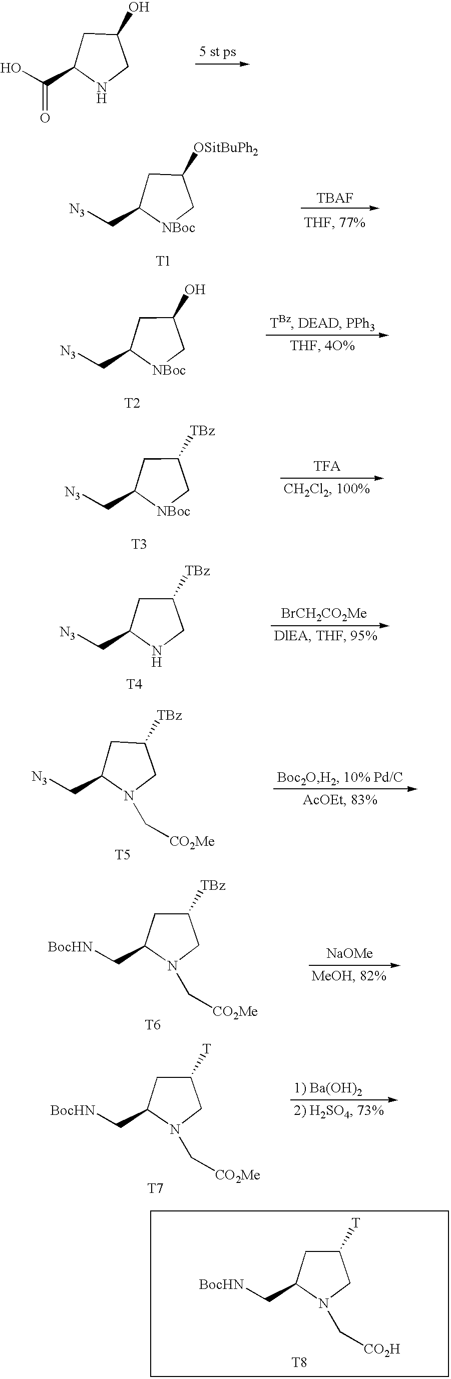

Preparation of compound T2. A 1 M solution of TBAF in THF (37 ml, 36.8 mmol) was added to a stirred solution of T1 (14.74 g, 30.7 mmol) in THF (150 ml) at room temperature. The reaction mixture was stirred 2 h at room temperature and then quenched by the addition of ¼ sat NH₄Cl (440 ml) and CH₂Cl₂ (600 ml). The layers were separated and the aq phase was extracted with more CH₂Cl₂ (600 ml) and AcOEt (250 ml). The combined organic phases were dried (MgSO₄) and the solvent was evaporated off. The crude product was purified by chromatography (50-100% AcOEt in heptane). Yield: 6.0 g (80%) of T2 as a white solid. NMR complicated by cis-trans isomeri around the Boc group: ¹H NMR (DMSO-d₆) δ 5.05 (d, J=3.1 Hz, 1H, D₂O exchangeable), 4.24-4.20 (m, 1H), 3.84 (br. s, 1H), 3.60-3.40 (m, 3H), 3.07 (br. s, 1H), 2.07 (br. s, 1H), 1.56 (br. s, 1H), 1.41 (s, 9H). ¹³C NMR (DMSO-d₆) δ 153.9, 153.4, 78.9, 68.8, 68.1, 56.0, 54.9, 54.7, 53.8, 52.7, 37.0, 36.2, 28.1. Calcd for C₁₀H₁₈N₄O₃: C, 49.56; H, 7.50; N, 23.13. Found: C, 49.65; H, 7.60; N, 22.83.

Preparation of compound T3. PPh₃ (8.98 g, 39.7 mmol) was added to a stirred solution of T2 (3.84 g, 15.9 mmol) and N-3-Benzoyl Thymine (7.32 g, 31.8 mmol) in dry THF (150 ml) at 0° C. The unclear solution was stirred 5 min before DEAD (6.25 ml, 39.7 mmol) was added dropwise at 0° C. during 1 h. The yellow reaction mixture was stirred at room temperature overnight before volatiles were evaporated off. The crude material was purified twice by chromatography.(50-66% AcOEt in heptane and 2-5% MeOH in CH₂Cl₂) to produce 4.23 g of T3. This material was further purified by recrystalization from AcOEt/heptane. Yield: 2.92 g (40%) of T3 as white needles. NMR complicated by cis-trans isomeri around the Boc group: ¹H NMR (DMSO-d₆) δ 7.98 (d, J=8.4 Hz, 2H), 7.76 (m, 2H), 7.59 (m, 2H), 5.15 (br. s, 1H) 4.13 (br. s, 1H), 3.67-3.38 (m, 4H), 2.44 (m, 1H), 2.12 (br. s, 1H), 1.85 (d, J=1.0 Hz, 3H), 1.43 (s, 9H). ¹³C NMR (DMSO-d₆) δ 169.7, 162.4, 149.5, 138.6, 135.4, 131.2, 130.4, 129.4, 109.4, 79.5, 55.4, 55.2, 53.6, 53.1, 52.6, 49.6, 49.4, 33.0, 32.0, 28.1, 12.1. Calcd for C₂₂H₂₆N₆O₅: C, 58.14; H, 5.77; N, 18.49. Found: C, 58.18; H, 5.69; N, 18.05.

Preparation of compound T5. TFA (4.95 ml, 64 mmol) was added to a stirred solution of T3 (2.92 g, 6.40 mmol) in dry CH₂Cl₂ (5.0 ml) at 0° C. The ice bath was removed and the reaction mixture was stirred at room temperature 45 min. The volatiles were evaporated off and the residue was evaporated from toluene to produce the TFA salt of T4. Yield: 3.60 g indicating the presence of some excess TFA. This material was dissolved in dry THF (32 ml) and DIEA (4.85 ml, 29 mmol) and then methyl bromoacetate (1.8 ml, 18.9 mmol) was added at 0° C. The ice bath was removed and the reaction mixture was stirred at room temperature overnight and then filtered through celite. The solvent was evaporated off and the crude product was purified by chromatography (2% MeOH in CH₂Cl₂). Yield: 2.24 g (94%) of T5 as a white solid. Pure on Tlc (R_{f}=0.36 using 2% MeOH in CH₂Cl₂ as eluent). ¹H NMR (DMSO-d₆) δ 7.96 (d, J=8.1 Hz, 2H), 7.87 (d, J=1.1 Hz, 1H), 7.77 (t, J=7.0 Hz. 2H), 7.59 (t, J=9.0 Hz, 2H), 4.93-4.88 (m, 1H), 3.71-3.63 (m,1H), 3.64 (s, 3H), 3.52-3.47 (m, 2H), 3.36-3.26 (m, 2H), 2.85-2.80 (t, J=8.6 Hz, 1H), 2.27-2.20 (m, 1H), 2.07-2.01 (m, 1H), 1.88 (d, J=1.1 Hz, 3H). ¹³C NMR (DMSO-d₆) δ 170.9, 169.8, 162.4, 149.5, 139.2, 135.4, 131.3, 130.4, 129.4, 109.2, 60.0, 56.0, 53.3, 52.4, 52.3, 51.4, 32.9, 12.1. Calcd for C₂₀H₂₂N₆O₅: C, 56.33; H, 5.20; N, 19.71. Found: C, 56.02; H, 5.09; N, 19.36.

Preparation of compound T6. A degassed solution of T5 (2.20 g, 5.16 mmol), Boc₂O (1.69 g, 7.74 mmol) and 10% Pd/C (0.22 g) in AcOEt (75 ml) was hydrogenated at room temperature overnight using balloon technique. Occasionally the nitrogen that developed was lead out through a needle outlet. The catalyst was removed by filtering the solution through celite. The solvent was evaporated off and the crude product was purified by chromatography (50-100% AcOEt in heptane). Yield: 2.13 g (83%) of T6 as a white foam. ¹H NMR (DMSO-d₆) δ 7.96 (d, J=8.6 Hz, 2H), 7.88 (s, 1H), 7.77 (t, J=8.6 Hz, 1H), 7.59 (t, J=8.2 Hz, 2H), 6.74 (t, J=5.5 Hz, 1H), 4.89-4.85 (m, 1H), 3.64 (s, 3H), 3.64-3.41 (m, 2H), 3.30 (m, 1H), 3.14-3.06 (m, 2H), 2.90-2.76 (m, 2H), 2.12-2.01 (m, 2H), 1.88. (s, 3H), 1.37 (s, 9H). ¹³C NMR. (DMSO-d₆) δ 171.1, 169.8, 162.5, 155.8, 149.5, 139.2, 135.4, 131.3, 130.4, 129.6, 109.3, 77.8, 60.7, 56.2, 53.2, 61.4, 42.0, 33.4, 28.3, 12.1. Calcd for C₂₅H₃₂N₄O₇.1/4H₂O: C, 58.92; H, 6.44; N, 11.00. Found: C, 59.25; H, 6.51; N, 10.57.

Preparation of compound T7. To a stirred solution of T6 (2.08 g, 4.16 mmol) in MeOH (33 ml) was added a solution of NaOMe in MeOH (1.0 M, 8.3 ml, 8.3 mmol) at 0° C. The solution was stirred 5 h at room temperature and then quenched by addition of half sat NH₄Cl (50 ml) and AcOEt (100 ml). The aq phase was extracted with more AcOEt (2×100 ml). The combined organic phases were dried (MgSO₄) and the solvent was evaporated off. The crude product was purified by chromatography (Eluent:AcOEt). Yield: 1.36 g (82%) of T7 as a white solid. ¹H NMR (CDCl₃, 300 MHz) δ 10.10 (s, 1H), 7.22 (s, 1H), 5.21 (br. s, 1H), 5.01 (t, J=7.6 Hz, 1H), 3.62 (s, 3H), 3.52-3.20 (m, 5H), 2.97-2.80 (m, 2H), 2.12-2.00 (m, 2H), 1.83 (s, 3H), 1.33 (s, 9H). ¹³C NMR (CDCl₃, 300 MHz) δ 170.8, 163.9, 155.9, 150.9, 137.3, 110.9, 78.9, 60.4, 56.3, 52.9, 51.4, 51.3, 40.9, 33.1, 28.0, 12.1. Calcd for C₁₈H₂₈N₄O₆: C, 54.53; H, 7.12; N, 14.13. Found: C, 54.45; H, 7.04; N, 13.88. FAB⁺MS: 397.10 (MH⁺).

Preparation of compound T8. A solution of Ba(OH)₂.8H₂O (199 mg, 0.63 mmol) in H₂O (8 ml) was added dropwise to T7 (168 mg, 0.42 mmol) dissolved in THF (8 ml) at 0° C. The ice bath was removed and the reaction mixture was stirred at room temperature 30 min. More H₂O (10 ml) was added and the THF was evaporated off. pH was adjusted to 2.5 by adding 4 N H₂SO₄ (0.35 ml, 0.70 mmol) to the unclear solution. BaSO₄ was removed by centrifugation. The acidic solution was decanted and then lyophilized. The lyophilization was repeated from MeOH (0.5 ml) and H₂O (10 ml) to produce 117 mg (73%) of T8 as a white powder. ¹H NMR (DMSO-d₆): δ 11.21 (s, 1H), 7.63 (s, 1H), 6.77 (br. s, 1H), 4.90-4.86 (m, 1H), 3.60-2.70 (m, 7H), 1.98-1.93 (m, 2H), 1.77 (s, 3H), 1.38 (s, 9H). Irradiation of H6-Thymine (δ 7.63) gives a NOE effect (2%) to H4 (δ 4.90-4.86) and irradiation of H4 gives a NOE effect (2%) to H6-Thymine. ¹³C NMR (DMSO-d₆) δ 171.9, 163.8, 155.9, 151.0, 138.0, 109.3, 77.8, 61.2, 56.3, 52.8, 52.1, 42.0, 33.2, 28.3, 12.2. Pure on Tlc (Butanol:Acetic acid: H₂O 4:1:1) R_{f}=0.38 (UV, ninhydrin reactive). HR FAB⁺MS: 383.1959 (MH⁺) (Calcd for C₁₇H₂₇N₄O₆: 383.1930).

### Example 4

Preparation of Thymine (T) Oligomers

Oligomer Synthesis. To compare the DNA and RNA recognition of the (2R, 4S)-pyrrolidine PNA analogue with the (2R, 4R)pyrrolidine PNA analogue published by Hickman et al.(29) and very recently also by Kumar et al. (30) a pentamer homo thymine oligomer was synthesized using standard PNA synthesis conditions (28):

| | |
|---|---|
| PNA 1164: | H-(T)₅-LysNH₂ |
| PNA 2121: | H-(T#)₅-LysNH₂ |

T#=(2R, 4S) pyrrolidine PNA Monomer T8

Solid phase synthesis of H-(T#)₅-LysNH₂ (PNA 2121). This pentamer was synthesized (6 μmol scale) by the usual in situ neutralization method using HBTU and DIEA on a Boc-Lys-(2-Cl-Z)-MBHA-PS resin (50 mg, loading 0.12 mmol/g): The novel monomer T# (7.6 mg, 15.8 μmol, 2.5 eq) was dissolved in DMF (160 μl). DIEA (15.6 μl, 90 μmol) was added and this solution was added to HATU (5.8 mg, 15.3 □mol) suspended in pyridine (80 μl). The solution was preactivated 2 min and then added to the resin. The coupling reaction was allowed to proceed for 0.5 h before the activated solution was drained out. A small amount of beads were subjected to the Kaiser test, which produced a yellow color indicating complete reaction. Synthesis and cleavage was continued the usual way. After ether precipitation the crude PNA was purified by RP-HPLC. MALDI-MS: 1468 (Calcd for MH⁺: 1467). Pure on RP-HPLC.

Binding. The binding of PNA 1164 and PNA 2121 to poly dA and poly A was studied by thermal denaturation (Tm, Table 4). Also listed are the values given by Hickman et. al. (29) The thermal stability results presented in Table 4 clearly indicate that a T₅-PNA-oligomer having (2R, 4S)-pyrrolidine backbone (PNA 2121) forms stronger complexes with both poly dA and poly A as compared to the (2R, 4R)-pyrrolidine isomer (entry 3) and also compared to the parent aminoethylglycine PNA (PNA 1164, entry 1). These complexes are ascribed to triplexes and further studies on (pyrimidine-purine mixed sequences) pyrrolidine PNAs are required to establish whether the present results can be extended to duplex structures and are generally valid. Such studies are now in progress.

TABLE 4
Melting temperatures (Tm values)^{a}

| Entry | Sequence | Poly dA | Poly A |
|---|---|---|---|
| 1 | PNA 1164 | 53.0 (45.5) | 64.0 (58.5) |
| 2 | PNA 2121 | 78.5 (44.0) | 79.0 (51.5) |
| 3 | T₅ (2R,4R) | 57 | 49 |
| | _{b} | | |

^{a}Tₘ = melting temperature (measured in medium salt buffer: 100 mM NaCl, 10 mM phosphate, 0.1 mM EDTA, pH = 7.0). Measured from 10 to 90 ° C. (values in parentheses are measured from 90 to 10° C). Heating rate: 0.5 K/min. UV absorbance measured at 254 nm.
^{b}Values from (29).

### Example 5

Preparation of 5-methylcytosine (mC) Monomer

The Z-protected 5-methylcytosine monomer was synthesized according to the following scheme:

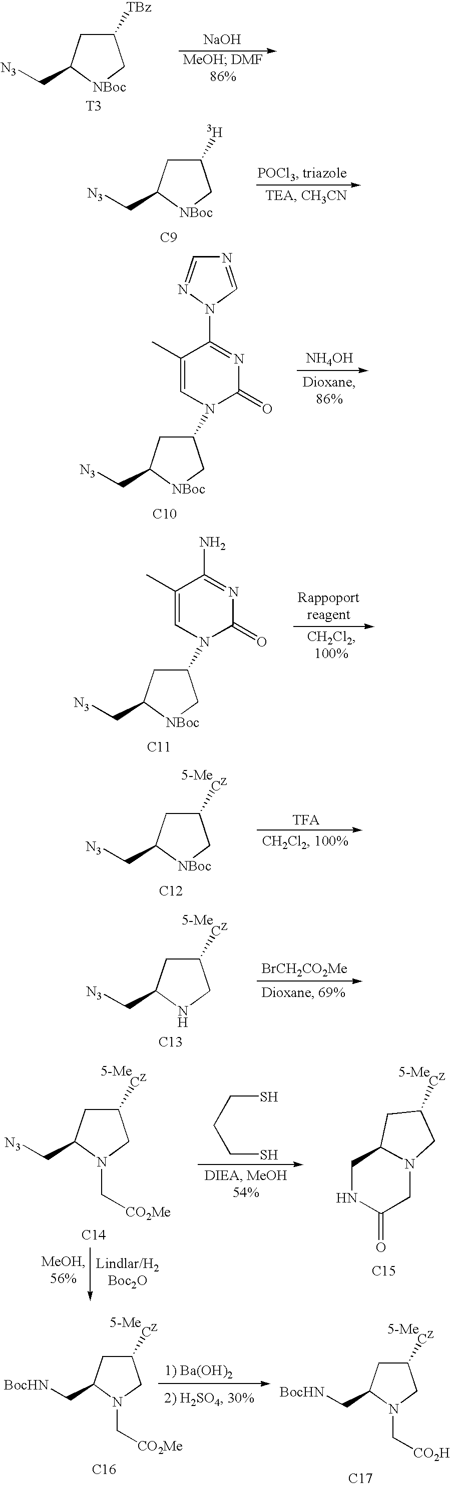

Preparation of compound C9. NaOH (2N, 8.7 ml, 17.4 mmol) was added to a stirred solution of compound T3 prepared as described in Example 3 (1.97 g, 4.33 mmol) in MeOH (35 ml) and DMF (25 ml) at 0° C. The solution was stirred 1.5 h at room temperature and then quenched by addition of half sat NaHCO₃ (50 ml) and AcOEt (250 ml). The aq phase was extracted with more AcOEt (250 ml). The combined organic phases were washed with brine (2×100 ml), dried (MgSO₄) and the solvent was evaporated off. The crude product was purified by chromatography (66-100% AcOEt in heptane). Yield: 1.31 g (86%) of C9 as a white solid. ¹H NMR (CDCl₃, 400 MHz) δ9.31 (s, 1H), 6.87 (s, 1H), 5.16 (t, J=6 Hz, H), 4.2-4.0 (m, 1H), 3.80-3.60 (m, 2H), 3.60-3.40 (m, 1H), 3.31 (m, 1H), 2.21 (m, 2H), 1.85 (s, 3H), 1.42 (s, 9H). FAB⁺MS: 351.4 (MH⁺).

Preparation of compound C11. A solution of C9 (1.30 g, 3.74 mmol) in dry CH₃CN (19 ml) was added dropwise to a stirred suspension of triazole (2.58 g, 37.4 mmol), Et₃N (5.2 ml, 37.4 mmol) and POCl₃ (0.7 ml, 7.48 mmol) in dry CH₃CN (19 ml) at 0° C. The solution was stirred at room temperature overnight. The solvent was evaporated off and the resulting solid was partitioned between sat NaHCO₃ (100 ml) and AcOEt (150 ml). The organic phase was washed with water (50 ml) brine (50 ml), dried (MgSO₄) and the solvent was evaporated off to produce 1.47 g (98%) of C10. Pure on tic: R_{f}=0.70 (CH₃OH/CH₂Cl₂: 1/9). FAB⁺MS: 402.4 (MH⁺). This material was dissolved in dioxane (14 ml) and conc. ammonia (4.7 ml) and stirred at room temperature 1 h. The solvent was evaporated off and the resulting solid was partitioned between sat NaHCO₃ (25 ml) and AcOEt (100 ml). The organic phase was washed with brine (50 ml), dried (MgSO₄) and the solvent was evaporated off. Yield: 1.13 g (86%) of C11 as a white solid. Pure on tic: R_{f}0.32 (CH₃OH/CH₂Cl₂: 1/9). ¹H NMR (DMSO-d₆, 300 MHz) δ 8.26 and 7.40 (2×s, 1H), 7.20 (s, 1H), 6.71 (s, 1H), 5.08 (m, 1H), 4.06 (br. s, 1H), 3.64-2.87 (m, 4H), 2.38 (m, 1H), 1.98 (m, 1H), 1.81 (s, 3H), 1.38 (s, 9H). Calcd for C₁₅H₂₃N₇O₃: C, 51.55; H, 6.65; N, 28.06. Found: C, 51.36; H, 6.52; N, 28.68. FAB⁺MS: 350.4 (MH⁺).

Preparation of compound C14. To a stirred solution of C11 (1.08 g, 3.09 mmol) in dry CH₂Cl₂ (21 ml) was added N-benzyloxycarbonyl-N′-methylimidazolium triflate (3.40 g, 9.3 mmol). The solution was stirred at room temperature overnight and then quenched by addition of sat NaHCO₃ (50 ml) and CH₂Cl₂ (50 ml). The aq phas was extracted with more CH₂Cl₂ (50 ml). The combined organic phases were dri d (MgSO₄) and then vacuated in vacuo. The crude product was purified by chromatography (2.5% CH₃OH in CH₂Cl₂). Yield: 1.57 g (100%) of C12 as a white solid. FAB⁺MS: 484.5 (MH⁺). Pure on tic: R_{f}=0.54 (2.5% CH₃OH in CH₂Cl₂). TFA (2.3 ml, 30 mmol) was added to a stirred solution of C12 (1.57 g) in dry CH₂Cl₂ (2.3 ml) at room temperature. The solution was stirred 2.5 h at room temperature. The solvent was evaporated off and the resulting oil was evaporated from a mixture of CH₃OH and toluene. This intermediate (C13 contaminated with excess TFA) was dissolved in dry THF (15 ml). DIEA (2.5 ml, 15 mmol) and then methyl bromoacetate (0.71 ml, 75 mmol) was added dropwise and the reaction mixture was stirred at room temperature 1.5 h. The solvent was evaporated off and the crude product was purified by chromatography (5% CH₃OH in CH₂Cl₂). Yield: 0.97 g (69%) of C14 as clear oil. Pure on ¹H NMR (DMSO-d₆, 300 MHz) δ 11.84 (s, 1H), 7.79 (s,1H), 7.37 (m, 5H), 5.10 (s, 2H), 4.92 (m, 1H), 3.62 (s, 3H), 3.50-3.23 (m, 6H), 2.76 (t, J=8.5 Hz, 1H), 2.17 (m, 1H), 1.99 (m, 1H) 1.84 (s, 3H). ¹³C NMR (DMSO-d₆) δ 170.8, 162.9, 159.1, 148.1, 139.8, 136.4, 128.3, 128.1, 127.9, 109.3, 66.7, 59.9, 55.9, 53.4, 52.3, 52.2, 51.3, 32.8, 12.9 FAB⁺MS: 456.4 (MH⁺).

Preparation of compound C16. A degassed solution of C14 (344 mg, 0.75 mmol), Boc₂O (327 mg, 1.50 mmol) and 5% Pd/CaCO₃/Pb (Lindlar) (75 mg) in MeOH (15 ml) was hydrogenated at room temperature 5 h using balloon technique. Occasionally the nitrogen that developed was lead out through a needle outlet. The catalyst was removed by filtering the solution through celite. The solvent was evaporated off and the crude product was purified by chromatography (66-100% AcOEt in heptane). Yield: 225 mg (56%) of C16 as a white foam. ¹H NMR (DMSO-d₆, 300 MHz) δ 12.13 (s, 1H), 7.36-7.21 (m, 6H), 5.11 (s, 2H), 5.06 (m, 2H), 3.64 (s, 3H), 3.45-2.87 (m, 7H), 2.10-1.96 (m, 2H), 1.92 (s, 3H), 1.37 (s, 9H). ¹³C NMR (DMSO-d₆) δ 170.7, 163.3, 160.4, 155.9, 148.1, 138.1, 135.8, 128.04, 128.01, 127.7, 111.0, 79.0, 67.2, 60.2, 56.2, 53.5, 51.3, 51.2, 40.9, 33.3, 28.0, 13.2. FAB⁺MS: 530.3 (MH⁺).

Preparation of compound C17. A solution of Ba(OH)₂.8H₂O (190 mg, 0.60 mmol) in H₂O (8 ml) was added dropwise to C16 (210 mg, 0.40 mmol) dissolved in THF (8 ml) at 0° C. The ice bath was removed and the reaction mixture was stirred at room temperature 30 min. More H₂O (10 ml) was added and the THF was evaporated off. pH was adjusted to 3 by adding 4 N H₂SO₄ (0.32 ml, 0.64 mmol) to the unclear solution. More H₂O (20 ml) was added before BaSO₄ was removed by centrifugation. The acidic solution was filtered and then lyophilized. The lyophilization was repeated from MeOH (1 ml) and H₂O (20 ml) to produce 62 mg (30%) of C17.H₂SO₄ as a whit powder. ¹H NMR (CDCl₃, 300 MHz) peaks shows considerabl broadening probably due to the presenc of H₂SO₄: δ 7.6 (br. s, 1H), 7.2 (m, 5H), 5.08 (s, 1H), 4.5-3.8 (m, 4H), 3.8-3.2 (m, 5H), 2.6-2.2 (m, 2H), 1.88 (s, 3H), 1.38 (s, 9H). Pure in Tlc (Butanol:Acetic acid:H₂_{O} 4:1:1) R_{f}=0.33 (UV, ninhydrin reactive).

### Example 6

Preparation of Guanine (G) Monomer

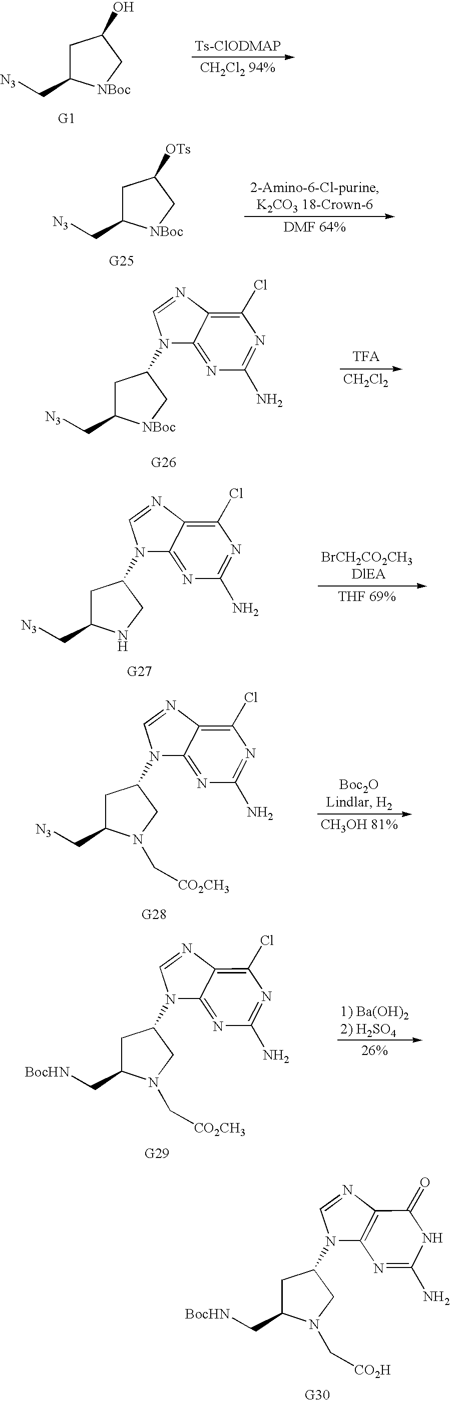

Preparation of compound G25. To a stirred solution of G1 (1.72 g, 7.10 mmol) in dry CH₂Cl₂ (70 ml) was added tosyl chloride (2.03 g, 10.6 mmol) and then DMAP (2.17 g, 17.8 mmol). The reaction mixture was stirred at rt overnight and then quenched by the addition of H₂O (150 ml) and CH₂Cl₂ (150 ml). The layers were separated and the aq phase was extracted with more CH2Cl₂ (100 ml). The combined organic phases were extracted with 10% citric acid (2×100 ml), brine (100 ml), sat NaHCO₃ (2×100 ml) and brine (100 ml). The organic phase was dried (Na₂SO₄) and the solvent was evaporated off. The crude product was purified by chromatography (AcOEt/heptane 1:2). Yield: 2.60 g (93%) of G25 as a clear oil. Pure on Tlc (AcOEt:heptane 1:2) R_{f}=0.32 (UV, ninhydrin reactive). NMR complicated by cis-trans isomeri around the Boc group: ¹H NMR (DMSO-d₆) δ 7.82 (d, J=8.4 Hz, 2H), 7.49 (d, J=9.0 Hz, 2H), 5.06 (m, 1H), 3.88 (m, 1H), 3.57 (m, 1H), 3.47 (m, 1H), 3.34 (m, 1H), 3.26 (m, 1H), 2.43 (s, 3H), 2.28 (m, 1H), 1.90 (m, 1H), 1.38 (s, 9H). ¹³C NMR (DMSO-d₆) δ 153.2, 145.2, 133.0, 130.3, 127.6, 80.2 and 79.5, 55.4, 53.2, 52.2, 34.5, 33.7, 28.0, 21.1. HR FAB⁺MS: 397.1540 (MH⁺) (Calcd for C₁₇H₂₅N₄O₅S: 397.1546).

Preparation of compound G26. A solution of G25 (2.60 g, 6.50 mmol) in dry DMF (17 ml) was added dropwise to a stirred suspension of 2-amino-6-chloropurine (1.60 g, 9.4 mmol), K₂CO₃ (1.35 g, 9.75 mmol) and 18-crown-6 (2.57 g, 9.75 mmol) in dry DMF (17 ml). The reaction mixture was stirred at 85° C. 2 h. The solvent was evaporated off and the residue was partitioned between H₂O (100 ml) and AcOEt (200 ml). The layers were separated and the aq phase was extracted with more AcOEt (100 ml). The combined organic phases were washed with brine (2×100 ml). The organic phase was dried (Na₂SO₄) and the solvent was evaporated off. The crude product (2.5 g) was purified by chromatography (2-5% MeOH in CH₂Cl₂). Yield: 1.65 g (64%) of G26 as a white solid. Pure on Tlc (AcOEt) R_{f}=0.61 (UV, ninhydrin reactive). Calcd for C₁₅H₂₀ClN₉O₂: C, 45.74; H, 5.13; N, 32.01. Found: C, 46.37; H, 5.23; N, 31.43. FAB⁺MS: 394.17 (MH⁺).

Preparation of compound G28. TFA (2.2 ml, 28.5 mmol) was added to a stirred solution of G26 (374 mg, 0.95 mmol) in dry CH₂Cl₂ (2.2 ml) at rt. The reaction mixture was stirred at rt 25 min. The volatile was evaporated off and the residue was evaporated from toluene to produce the TFA salt of G27. Yield: 735 mg indicating the presence of some excess TFA. This material was dissolved in dry THF (4.5 ml) and DIEA (0.83 ml, 4.75 mmol) and then methyl bromoacetate (0.11 ml, 1.14 mmol) was added at rt. The reaction mixture was stirred at rt overnight and then filtered through celite. The solvent was evaporated off and the crude product was purified by chromatography (AcOEt). Yield: 241 mg (69%) of G28 as a white solid. ¹H NMR (DMSO-d₆) δ 8.24 (s, 1H), 6.89 (s, 2H), 4.90 (m, 1H), 3.80-3.20 (m, 6H), 3.61 (s, 3H), 2.95 (t, J=9.0 Hz, 1H), 2.44 (m, 1H), 2.19 (m, 1H). ¹³C NMR (DMSO-d₆) δ 170.8, 159.6, 154.0, 149.4, 141.3, 123.5, 59.9, 57.3, 52.6, 52.3, 51.6, 51.3, 33.7. Calcd for C₁₃H₁₆ClN₉O₂: C, 42.68; H, 4.42; N, 34.47. Found: C, 43.02; H, 4.39; N, 34.14. HR FAB⁺MS: 366.1197 (MH⁺) (Calcd for C₁₃H₁₇ClN₉O₂: 366.1194).

Preparation of compound G29. A degassed solution of G28 (718 mg, 1.96 mmol), Boc₂O (872 mg, 4 mmol) and 5% Pd/CaCO₃/Pb (Lindlar) (700 mg) in CH₃OH (40 ml) was hydrogenated at rt 2 h using balloon technique. Occasionally the nitrogen that developed was lead out through a needle outlet. The catalyst was removed by filtering the solution through celite. The solvent was evaporated off and the crude product was purified by chromatography (0-10% MeOH in AcOEt). Yield: 702 mg (81%) of G29 as a white solid. ¹H NMR (DMSO-d₆) δ 8.25 (s, 1H), 6.88 (s, 2H), 6.74 (t, J=5.5 Hz, 1H), 4.86 (m, 1H), 3.62 (s, 3H), 3.65-3.10 (m, 5H), 2.91 (t, J=8.3 Hz, 2H), 2.34 (m, 1H), 2.10 (m, 1H), 1.37 (s, 9H). ¹³C NMR (DMSO-d₆) δ 171.6, 159.6, 155.8, 154.0, 149.4, 141.4, 123.6, 77.8, 60.4, 57.6, 52.5, 51.5, 51.3, 42.7, 34.2, 28.3. Calcd for C₁₈H₂₆ClN₇O₄.1/2H₂O: C, 48.15; H, 6.07; N, 21.84. Found: C, 48.44; H, 5.82; N, 21.29. HR FAB⁺MS: 440.1816 (MH⁺) (Calcd for C₁₈H₂₇ClN₇O₄: 440.1813).

Preparation of compound G30. A solution of Ba(OH)₂.8H₂O (146 mg, 0.46 mmol) in H₂O (11 ml) was added dropwise to G29 (155 mg, 0.353 mmol) dissolved in THF (11 ml) at rt. The reaction mixture was stirred at rt overnight. The reaction was followed with Tlc (Butanol:Acetic acid: H₂O 4:1:1): As expected the methylester was cleaved in less than 15 min but no slow formation of G30 could be detected (corresponding to displacing of the chloro atom by hydroxide). More Ba(OH)₂.8H₂O (146 mg, 0.46 mmol) in H₂O (11 ml) was added and the solution was reluxed overnight and the 3 days at rt. The THF was evaporated off. pH was adjusted to 3 by adding 4 N H₂SO₄ (0.49 ml) to the unclear solution. More H₂O (25 ml) was added before BaSO₄was removed by centrifugation. The acidic solution was filtered and then lyophilized. After lyophilization the crude product was purified by chromatography (RP-18) using a gradient of 0-50% MeOH In H₂O. Yield: 37 mg (26%) of G30 as a white solid. Pure on Tlc (Butanol:Acetic acid: H₂O 4:1:1) R_{f=}0.40 (UV, ninhydrin reactive). ¹H NMR (DMSO-d₆) δ 10.58 (s, 1H), 7.83 (s, 2H), 6.74 (s, 1H), 6.44 (s, 2H), 4.75 (m, 1H), 3.60-3.20 (m, 3H), 3.15 (m, 2H), 2.90 (m, 2H), 2.26 (m, 1H), 2.10 (m, 1H), 1.37 (s, 9H). ¹³C NMR (DMSO-d₆) δ 172.0, 156.0, 155.8, 153.4, 151.1, 135.3, 116.7, 77.7, 60.5, 58.0, 52.9, 50.8, 42.6, 34.3, 28.2. Calcd for C₁₇H₂₆N₇O₅.5/2H₂O: C, 45.12; H, 6.70; N, 21.67.. Found: C, 44.97; H, 6.27; N, 21.31. HR FAB⁺MS: 440.1990 (MH⁺) (Calcd for C₁₇H₂₆N₇O₅: 440.1995).

### Example 7

Stability of pyr-PNA Oligomer

The binding properties of pyr-PNA oligomer consisting of 10 monomers of the following sequence: H-CTC ATA CTC T-Lys-NH2 were investigated by measuring the thermal stability of the complexes formed with sequence complementary DNA and RNA, respectively, as compared to the stability for the corresponding aeg-PNA oligomer.

The stability expressed as the melting temperature (Tₘ), defined as the temperature at which 50% of the complexes have been dissociated, was determined as described by Arghya Ray et al (32).

The following results (Tₘ, ° C.) were obtained:

| | DNA antiparallel¹ | | DNA parallel³ | | RNA antiparallel⁴ | |
|---|---|---|---|---|---|---|
| PNA¹ | up | down | up | down | up | down |
| PNA 2244⁵ | 45.0° | (43.0°) | 44.0° | (43.5°) | n.m. | |
| PNA 2232⁶ | 53° + 71.0° | (58.0°) | 56.5° + 73.5° | (61.5°) | 40.5° + 65.5° | (41.0°) |

Measured in medium salt buffer: 100 mM NaCl, 10 mM Na-phosphate, 0.1 mM EDTA, pH=7.0.

| | |
|---|---|
| 1: PNA sequence: | H-CTC ATA CTC T-Lys-NH2 |
| 2: DNA antiparallel: | 5′-dAGA GTA TGA GTA-3′, |
| 3: DNA parallel: | 5′-dATG AGT ATG AGA-3′ |
| 4: RNA antiparallel: | 5′-AGA GUA UGA GUA-3′ |
| 5: aeg PNA | |
| 6: pyr PNA | |

1. A peptide nucleic acid (PNA) oligomer characterized in that the single units of the oligomer consists of different amino acid backbones selected from aminoethylglycine (aeg), aminoethylprolyl (aep), aminoethylpyrrolidine (pyr) or an amino acid other than aeg, aep and pyr.

2. A peptide nucleic acid oligomer of claim 1 with from 4 to 25 monomers selected from the group consisting of aeg-PNA, pyr-PNA, aep-PNA or aa-PNA, provided that the oligomer contains at least one pyr-PNA monomer group.

3. A peptide nucleic acid oligomer of claim 1 or 2 wherein all monomers are selected from pyr-PNA.

4. A peptide nucleic acid oligomer of claim 1 or 2 wherein the monomers are selected from pyr-PNA and aeg-PNA.

5. A peptide nucleic acid oligomer of the above claims wherein the pyr-PNA is a compound of formula II 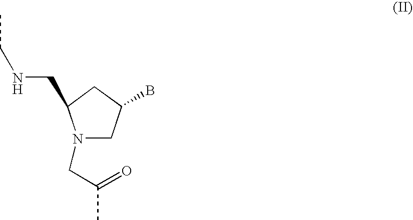
wherein B is a nucleobase selected from naturally occurring or non-naturally occurring nucleobases.

6. A modified PNA molecule of formula (I):
Q—L—PNA (I)
wherein L is a linker or a bond;
Q is a peptide and
PNA is a peptide nucleic acid oligomer of claim 1 or 2.

7. A modified PNA molecule of formula (I):
Q—L—PNA (I)
wherein L is a linker or a bond;
Q is a peptide and
PNA is a peptide nucleic acid oligomer of claim 3.

8. A modified PNA molecule of formula (I):
Q—L—PNA (I)
wherein L is a linker or a bond;
Q is a peptide and
PNA is a peptide nucleic acid oligomer of claim 4.

9. Use of a compound of any of the claims 1 to 8 for down regulation of the expression of specific genes by targeting the genes at the mRNA or at the DNA level.

10. A method of treating a disease selected from bacterial and viral infections, cancer, metabolic diseases or immunological disorders comprising administering to a patient in need thereof an efficient amount of a compound of claim 1 to 8.

11. Use of a compound of claim 1 to 8 in the manufacture of a medicament.

12. Use according to claim 11 in the manufacture of a medicament for the treatment of bacterial or viral infections, cancer, metabolic diseases or immunological disorders.

13. Us of a compound of claim 1 to 8 as a hybridization probe in genetic diagnostics.

14. Use according to claim 12, selected from in situ hybridization, real time PCR monitoring or PCR modulation by “PNA-clamping”.

17. A peptide nucleic acid (PNA) oligomer, wherein each monomer consists of a different amino acid backbone selected from aminoethylglycine (aeg), aminoethylprolyl (aep), aminoethylpyrrolidine (pyr), or an amino acid other than aeg, aep and pyr (aa).

18. A peptide nucleic acid oligomer of claim 17, comprising 4 to 25 monomers selected from the group consisting of aeg-PNA, pyr-PNA, aep-PNA or aa-PNA, wherein the oligomer contains at least one pyr-PNA monomer.

19. A peptide nucleic acid oligomer of claim 17, wherein each monomer is pyr-PNA or aeg-PNA.

20. A peptide nucleic acid oligomer of claim 17, wherein each monomer is pyr-PNA

21. A peptide nucleic acid oligomer of claim 17 wherein the pyr-PNA is a compound of formula II: 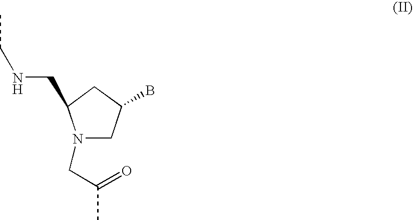
and wherein B is a nucleobase selected from naturally occurring or non-naturally occurring nucleobases.

22. A modified PNA molecule of formula (I):
Q—L—PNA (I)
wherein L is a linker or a bond;
Q is a peptide; and
PNA is a peptide nucleic acid oligomer of claim 17.

23. A modified PNA molecule of formula (I):
Q—L—PNA (I)
wherein L is a linker or a bond;
Q is a peptide; and
PNA is a peptide nucleic acid oligomer of claim 19.

24. A modified PNA molecule of formula (I):
Q—L—PNA (I)
wherein L is a linker or a bond;
Q is a peptide and
PNA is a peptide nuleic acid oligomer of claim 20.

25. A method for down regulation of the expression of a nucleic acid, comprising contacting a cell with a PNA oligomer of claim 17 which comprises a sequence that is complementary to, and binds to, at least one nucleic acid sequence in the cell.

26. The method of claim 25, wherein the nucleic acid is DNA or mRNA.

27. A method of treating a disease selected from a bacterial or viral infection, cancer, a metabolic diseases or an immunological disorder, comprising administering to a patient in need thereof an effective amount of a PNA oligomer of claim 17 and a pharmaceutically acceptable carrier, wherein the compound comprises a sequence that is complementary to, and binds to, at least one nucleotide sequence in the cell that is associated with said disease.

28. A composition comprising a PNA oligomer of claim 17, or a pharmaceutically acceptable salt thereof, and a pharmaceutically acceptable carrier.

29. A composition of claim 28, further comprising a transporter polymer to enhance transport across a biological membrane.

30. A method for determining the presence of a target nucleotide sequence in a cell comprising (i) contacting at least one nucleic acid from the cell with a PNA oligomer of claim 17, wherein the compound comprises a sequence that is complementary to the target nucleotide sequence, and (ii) detecting binding of the PNA oligomer to the target sequence.

31. The method of claim 30, wherein the method is selected from in situ hybridization, real-time PCR, or PNA-clamping.
